(19) United States
(12) Patent Application Publication
(10) Pub. No.: US 2013/0302812 A1
(43) Pub. Date: Nov. 14, 2013

(54) METHOD OF MICROALGAL CHLOROPLAST TRANSFORMATION USING FUNCTIONAL SELECTION IN THE ABSENCE OF ANTIBIOTICS

(71) Applicants: Anastasios Melis, El Cerrito, CA (US); Hsu-Ching Chen Wintz, El Cerrito, CA (US)

(72) Inventors: Anastasios Melis, El Cerrito, CA (US); Hsu-Ching Chen Wintz, El Cerrito, CA (US)

(73) Assignee: The Regents of the University of California, Oakland, CA (US)

(21) Appl. No.: 13/883,812

(22) PCT Filed: Nov. 7, 2011
(86) PCT No.: PCT/US11/59636

§ 371 Date: Jul. 23, 2013

Related U.S. Application Data
(60) Provisional application No. 61/411,387, filed on Nov. 8, 2010.

(51) Int. Cl. C12N 15/82

(52) U.S. Cl. CPC C12N 15/8241
USPC 435/6.12; 435/257.2

(57) ABSTRACT

This invention relates to a new method of selecting for transgene transformants in the absence of antibiotic selective pressure, where the method is based on recovery of function.

### CROSS-REFERENCE TO RELATED APPLICATIONS

This application claims benefit of priority to U.S. provisional application No. 61/411,387 filed Nov. 8, 2010, which application is herein incorporated by reference for all purposes.

### BACKGROUND OF THE INVENTION

Chloroplast transformation in green microalgae relies on the use of antibiotics as selectable marker for the isolation of transformants. For example, a streptomycin resistance cassette as a selectable marker is commonly used. However, isolation of transformants based on antibiotic resistance selectable marker is subject to various pitfalls: (i) the method is subject to isolating multiple false positives; (ii) there is a low efficiency of transformation, or co-transformation based on streptomycin and transgene transformation; (iii) there is difficulty in attaining transgenic chloroplast DNA copy segregation (homoplasmy) under antibiotic resistance pressure; and (iv) there are environmental and monetary difficulties in using the selectable marker (antibiotics) under mass culture and commercial production conditions.

All of the above pitfalls are eliminated with the “recovery of function” selectable marker of the invention. Superior transformation results (100% positive transformants) are obtained with this “recovery of function” method, than with the streptomycin-based transformation of C. reinhardtii that is currently used in the field. High yield of transgene expression is achieved with this method. Moreover, high yield of product generation was achieved under defined conditions with the “recovery of function” method, i.e. 30-100 fold better than those achieved with traditional approaches.

### BRIEF SUMMARY OF THE INVENTION

This invention is based, in part on the discovery that recovery of photosynthetic function can be used as a selectable marker, without antibiotic selection, in the transformation of microalgal chloroplasts. This method alleviates the need to use antibiotics for transformant strain selection, results in very low levels, e.g., (0%), false positives during screening, and further ensures a directed segregation of chloroplast DNA so as to unequivocally achieve homoplasmy in all copies of the transformant chloroplast DNA, with a concomitant elimination of wild type DNA copies. This invention also permits the generation of transgenic microalgal strains that do not contain antibiotic resistance gene(s), thus alleviating concerns of genetically engineered organisms in industrial application.

### BRIEF DESCRIPTION OF THE DRAWINGS

FIG. 1. P-67 cpDNA EcoRI 14. Strain: CC-125 wild type mt+137c. Insert: chloroplast DNA EcoRI 14 (5.5 kb). Genes present: PsaB, RbcL, AtpA (5′). Vector: pUC8; bacterial host strain: JM83; selectable marker: amp-r, white on X-gal. (From the http://website www.chlamy.org, see also Boynton and Gillham 1983).

FIG. 2. Construction of expression vector for Chlamydomonas reinhardtii chloroplast transformation. (A) P-67 Insert: chloroplast DNA EcoRI 14 (5.8 kb). Genes present: PsaB, RbcL, AtpA (5′). Vector: pUC8; (selectable marker: amp-r (From Boynton and Gillham 1983). (B) Cloning of the EcoRI 14 (5.8 kb) into pGEMT-easy vector (Promega), named pGEMT67. (C) Site-directed mutagenesis of pGEMT67, introducing a unique SmaI restriction site in the intergenic region of PsaB-tRNAG, plasmid named pGEMT67Sma.

FIG. 3. Sequence alignment of ADH1 gene from Saccharomyces cerevisiae (ScADH1) and the Chlamydomonas reinhardtii chloroplast codon optimized ADH gene (CrCpADH). Translation initiation codon ATG and termination codon TAA are indicated in bold-underlined font. The 6×His tag at the 3′ end of the CrCpADH gene is underlined.

FIG. 4. Sequence of the CrCpADH expression cassette for Chlamydomonas chloroplast transformation. The RbcL promoter employed, including the first 90 nucleotides of the RbcL coding sequence, is indicated in italic-bold-underlined font. The CrCpADH coding sequence is from ATG to TAA. Initiation and termination codons of the ADH gene are shown in shaded and underlined font. Primers used for the RT-PCR experiments are indicated as forward (CrCpADHF1,2) or reverse (CrCpADHR1,2) and the corresponding nucleotide sequences are underlined. The RbcL terminator sequence is indicated in italic font.

FIG. 5. Map of the expression vector pHCCrCpADH used for Chlamydomonas chloroplast transformation and detailed composition of the CrCpADH expression cassette. The CrCpADH expression cassette was cloned into the SmaI site of pGEMT67Sma plasmid. This resulting plasmid is named pHCCrCpADH. RbcLp: RbcL promoter; RbcL90: the first 90 nucleotides of the RbcL gene coding sequence; CrCpADH: Chlamydomonas reinhardtii chloroplast codon optimized ADH gene sequence; 6×His-tag was included at the 3′ end of the CrCpADH coding sequence; RbcLt:RbcL terminator.

FIG. 6. Map of the expression vector pHCCrCpPDC used for Chlamydomonas chloroplast transformation and detailed composition of the CrCpPDC expression cassette. The CrCpPDC expression cassette was cloned into the SmaI site of pGEMT67Sma plasmid. This resulting plasmid is named pHCCrCpPDC. RbcLp: RbcL promoter; RbcL90: the first 90 nucleotides of the RbcL gene coding sequence; CrCpPDC: Chlamydomonas reinhardtii chloroplast codon optimized ADH gene sequence; 6×His-tag was included at the 3′ end of the CrCpPDC coding sequence; RbcLt: RbcL terminator.

FIG. 7. Map of the expression vector pHCCrCpIspS used for Chlamydomonas chloroplast transformation and detailed composition of the CrCpIspS expression cassette. The CrCpIspS expression cassette was cloned into the SmaI site of pGEMT67Sma plasmid. This resulting plasmid is named pHCCrCpIspS. RbcLp: RbcL promoter; RbeL90: the first 90 nucleotides of the RbcL gene coding sequence; CrCpIspS: Chlamydomonas reinhardtii chloroplast codon optimized ADH gene sequence; 6×His-tag was included at the 3′ end of the CrCpIspS coding sequence; RbcLt: RbcL terminator.

FIG. 8. Schematic presentation of homologous recombination during Chlamydomonas chloroplast transformation. Double homologous recombination occurs between the endogenous (CC2653) and plasmid (pHCCrCpADH) PsaB and tRNAG-RbcL DNA regions, resulting in the replacement of the mutated endogenous RbcL gene sequence (the mutation is marked by a vertical red line) of the recipient strain (CC2653) with a functional copy of the RbcL gene located on the expression vector pHCCrCpADH. The double homologous recombination is also accompanied by a concomitant insertion of the CrCpADH gene in the chloroplast DNA. An example of a double homologous recombination product is shown in joining three yellow arrow bars. The resulting recombinant product contains wild type sequences of PsaB and tRNAG-RbcL DNA, and also the CrCpADH expression cassette.

FIG. 9. Illustration of the endogenous chloroplast DNA EcoRI 14 fragment in strain CC2653 and map of the transgenic DNA organization following chloroplast transformation with plasmid carrying the CrCpADH construct. The location of the primers used for testing homoplasmy is indicated by red (forward primer) and black (reverse primer) bars. The expected PCR product sizes are also indicated, when using the specified primers (red and black boxes).

FIG. 10. Analysis of eight chloroplast transformant colonies from different single-cell lines that grew under selective (photoautotrophic) conditions. (A) Lanes 1 through 8 show the genomic PCR products of eight independent trangenic Chlamydomonas lines. M: 1 kb plus ladder (Invitrogen) used as molecular size marker. Lane 9: strain CC2653 mating type test by genomic DNA PCR using mating type “+” specific primers; lane 10: strain CC2918 mating type test by genomic DNA PCR using mating type “−” specific primers. Lanes 11 and 12 show the negative PCR results of CC2653 (11) and CC2918 (12) when using CrCpADH specific primers. (B) Homoplasmy test of the eight Chlamydomonas chloroplast transformant lines, shown in (A) above, by genomic DNA PCR using specific primers as indicated in FIG. 4.

FIG. 11. Chlamydomonas reinhardtii chloroplast transformants RT-PCR analysis. CrCpADH transgene expression in eight independent single-cell lines (CpT-0 through CpT-7) chloroplast transformants were analyzed. The top panel shows the RT-PCR results using CrCpADH gene specific primers, and the lower panel shows the RT-PCR results using RbcL specific primers.

FIG. 12. SDS-PAGE and Western blot analysis of Chlamydomonas reinhardtii chloroplast transformants. The left panel shows the Coomassie-blue stained SDS-PAGE protein profiles of three independent single-cell lines. Arrows indicate the electrophoretic mobility position of the large (LSU) and small subunit (SSU) of Rubisco. The right panel shows the Western blot ADH analysis results of three CpT strains with monoclonal antibodies directed against the His-tag, followed and chemiluminescence detection.

FIG. 13. Photosynthetic ethanol production and quantification analysis. (A) Ethanol calibration curve obtained with the dichromate-based analytical method (Ethanol Assay kit from BioChain) following the manufacturer's instructions. (B) and (C) Two sets of independent experiments of ethanol production analysis with CrCpADH transformants: CpT-0 to CpT-7 denote different lines of CrCpADH transformants. WT: wild type control. Experimental protocols employed are described in detail in the Materials and methods section. LD: light:dark cycle; D: dark incubation.

FIG. 14. Example of gas sample analysis from the headspace of CrCpIspS transformed Chlamydomonas chloroplast. The profile of gases from the headspace shows a prominent isoprene peak, with an elution time of 4-5 min.

FIG. 15. Homoplasmy test of the five CrCpIspS transformed Chlamydomonas lines, by genomic PCR using specific primers as indicated in FIG. 4. The presence of the 292 bp fragment indicated the integration of the CrCpIspS trangene expression cassette into the chloroplast genome of Chlamydomonas. The absence of the 413 bp indicated the attainment of the homoplasmic state of the chloroplast genomes in the transformant.

FIG. 16. Analysis of CrCpPDC transformed Chlamydomonas lines. (A) Homoplasmy test of the six transformant lines, by genomic PCR using specific primers as indicated in FIG. 4. Amplification of the 292 bp fragment indicated the integration of the CrCpPDC trangene expression cassette into the chloroplast genome of Chlamydomonas. The absence of the 413 bp indicated the attainment of the homoplasmic state of the chloroplast genomes in the transformant. (B) Western blot analysis of three transformants lines, using specific polyclonal antibodies raised against purified PDC. The results showed specific crossreactions of the antibodies with a 70 kD protein which corresponds to the predicted size of the transgenic PDC protein.

### DETAILED DESCRIPTION OF THE INVENTION

### Definitions

As used herein, the term “photoautotrophic growth conditions in minimal media” refers to culture conditions in which the carbon source for microalgal cell growth is provided by photosynthesis.

In the context of this invention, a “photosynthetic nucleic acid” or “photosynthetic gene” or “photosynthetic polynucleotide” refers to a gene that plays an essential role in photosynthesis such that inactivation of the gene, so that no functional protein product of the gene is generated, results in the loss of the ability to grow in the absence of media supplementation with an organic carbon source, e.g., acetate. Thus, mutation of such a photosynthetic gene is lethal in the absence of an external organic carbon source present in the growth media. In the present invention, a photosynthetic polynucleotide or a fragment thereof is introduced into a microalgal cell that has a lethal mutation in that photosynthetic gene. The gene introduced into the cell can encode the photosynthesis protein itself, e.g., it may be a cDNA sequence or chloroplast genomic DNA sequence that encodes the photosynthesis protein, or it may be a fragment of the photosynthetic gene that provides for restoration of function, e.g., through homologous recombination, once the polynucleotide is introduced into the mutant algal cells.

As used herein, microalgae refers to green microalgae and blue-green microalgae (cyanobacteria). Examples of green microalgae include Chlamydomonas reinhardtii, Scenedesmus obliquus, Chlorella vulgaris, Botryococcus braunii, Botryococcus sudeticus, Dunaliella salina, and Haematococcus pluvialis. Examples of cyanobacteria include Synechocystis sp. and Synechococcus sp.

The terms “nucleic acid” and “polynucleotide” are used synonymously and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones. Thus, nucleic acids or polynucleotides may also include modified nucleotides, that permit correct read through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc

The phrase “a nucleic acid sequence encoding” refers to a nucleic acid which contains sequence information for a structural RNA such as rRNA, a tRNA, or the primary amino acid sequence of a specific protein or peptide, or a binding site for a trans-acting regulatory agent. This phrase specifically encompasses degenerate codons (i.e., different codons which encode a single amino acid) of the native sequence or sequences that may be introduced to conform with codon preference in a specific host cell. In the context of this invention, the term “coding region” when used with reference to a nucleic acid reference sequence refers to the region of the nucleic acid that encodes the protein.

A polynucleotide sequence is “heterologous to” a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from any naturally occurring allelic variants.

A “transgene” is a sequence encoding a protein of interest that is to be expressed in microalgal cells. A “transgene” is heterologous to the host microlagal cells

An “expression cassette” refers to a nucleic acid construct, which when introduced into a host cell, results in transcription and/or translation of a RNA or polypeptide, respectively. Often such an expression cassette comprises a transgene operatively linked to a promoter; however, in some embodiments, expression of a transgene present in the expression cassette may be driven by an endogenous promoter.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the term “transgene polynucleotide sequence” or “transgene”.

The term “isolated”, when applied to a nucleic acid or protein, denotes that the nucleic acid or protein is essentially free of other cellular components with which it is associated in the natural state. It is preferably in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest.

Achieving homoplasmy” refers to a quantitative replacement of most, e.g., 70% or greater, or typically all, wild-type copies of the microalgal DNA in the cell with the transformant DNA copy that carries the transgene. This is normally attained over time, under the continuous selective pressure conditions applied, and entails the gradual replacement during growth of the wild-type copies of the DNA with the transgenic copies. Achieving homoplasmy is typically verified by quantitative amplification methods such as genomic-DNA PCR using primers and/or probes specific for the wild type copy of the microalgae chloroplast DNA. Transgenic DNA is typically stable under homoplasmy conditions and present in all copies of the chloroplast DNA.

### Introduction

The invention is based, in part, on the discovery that a selection procedure based on restoration of chloroplast function can be used to select transformed microalgal cells into which a transgene of interest has been introduced. The microalgae cells that are transformed in accordance with the methods of the invention have a defective chloroplast gene involved in photosynthesis such that the cells cannot survive in the absence of an external carbon source. Such microalgae cells are transformed with an expression cassette encoding a gene that restores photosynthetic ability. The expression cassette also encodes a transgene of interest to be expressed by the microalgae. Selection of transformants is performed in the absence of antibiotic selection.

The invention employs various routine recombinant nucleic acid techniques. Generally, the nomenclature and the laboratory procedures in recombinant DNA technology described below are those well known and commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel et al., eds., 1994-1999).

### Photosynthetic Gene/Mutants

Examples of microalga chloroplast DNA mutants and strains that can be utilized in the context of this invention, i.e., using restoration of chloroplast photosynthetic function for selection of transformants expressing a transgene of interest and to achieve DNA copy segregation (achieving homoplasmy), are those mutants that have a defect in, e.g., a point mutation, deletion, etc in a chloroplast gene that encodes a protein that is important for photosynthesis, thereby rendering them unable to grow autotrophically. Thus these microalgal mutants are photosynthesis lethal mutants. Examples of mutants in which chloroplast genes essential for photosynthesis are missing include:
ΔrbcL mutants, which are unable to assemble the RbcL large subunit of Rubisco. These mutants cannot fix CO₂ and require acetate for growth. (see, e.g., Spreitzer R J, Goldschmidt-Clermont M, Rahire M, Rochaix J D (1985) Nonsense mutations in the Chlamydomonas chloroplast gene that codes for the large subunit of ribulose-bisphosphate carboxylase/oxygenase. Proc Natl Acad Sci USA 82: 5460-5464; White A L, Melis A (2006) Biochemistry of hydrogen metabolism in Chlamydomonas reinhardtii wild type and a Rubisco-less mutant. Intl. J. Hydrogen Energy 31: 455-464).
ΔpsbA mutants, which are unable to assemble the psbA/D1 reaction center protein of photosystems-II and cannot perform photosystem-II photochemistry. These mutants require acetate for growth. (Bennoun P, Spierer-Herz M, Erickson J, Girard-Bascou J, Pierre Y, Delosme M, Rochaix J-D (1986) Characterization of photosystem II mutants of Chlamydomonas reinhardtii lacking the psbA gene. Plant Mol Biol 6:151-160; Dewez D, Park S, García-Cerdán J G, Lindberg P, Melis A (2009) Mechanism of the REP27 protein action in the D1 protein turnover and photosystem-II repair from photodamage. Plant Physiol. 151:88-99.
ΔpsbD mutants, which are unable to assemble the psbD/D2 reaction center protein of photosystems-II. Cannot do photosystem-II photochemistry. These mutants require acetate for growth. (Erickson J M, Rahire M, Malnoe P, Girard-Bascou J, Pierre Y, Bennoun P, and Rochaix J-D (1986) Lack of the D2 protein in a Chlamydomonas-reinhardtii psbD mutant affects photosystem-II stability and D1 expression; EMBO J. 5:1745-1754; Dewez D, Park S, García-Cerdán J G, Lindberg P, Melis A (2009) Mechanism of the REP27 protein action in the D1 protein turnover and photosystem-II repair from photodamage. Plant Physiol. 151:88-99).
ΔpetA mutants, which are unable to assemble the cytochrome f part of the cytochrome b-f complex. Cannot transport electrons between photosystem-II and photosystem-I. These mutants require acetate for growth. (Bertch J, Malkin R (1991): Nucleotide-sequence of the petA (cytochrome-f) gene from the green-alga, Chlamydomonas reinhardtii. Plant Mol Biol 17:131-133)
ΔatpA or ΔatpB mutants, which are unable to assemble the ATP synthase CF1 subunit. Cannot synthesize ATP. These mutants require acetate for growth. (Fiedler H R, Schlesinger J, Strotmann H, Shavit N, Stefan Leu S (1997) Characterization of atpA and atpB deletion mutants produced in Chlamydomonas reinhardtii cw15: Electron transport and photophosphorylation activities of isolated thylakoids. Biochim. Biophys. Acta 1319: 109-118).

Photosynthetic genes are known in the art. A photosynthetic nucleic acid sequence for use in the invention that is introduced into the mutant algal cells (that are mutant in the photosynthetic gene) can be a partial sequence or can encode functional protein itself. For example, a partial nucleotide sequence can be employed where recombination with the endogenous gene restores the ability of the gene to produce a functional protein.

In other embodiments, the photosynthetic gene may encode the functional protein.

As appreciated by one of skill in the art, expression constructs can be designed taking into account such properties as codon usage frequencies of the organism in which the expression construct nucleic acid is to be expressed. Codon usage frequencies can be tabulated using known methods (see, e.g., Nakamura et al. Nucl. Acids Res. 28:292, 2000). Codon usage frequency tables, including those for microalgae and cyanobacteria, are also available in the art (e.g., in codon usage databases of the Department of Plant Genome Research, Kazusa DNA Research Institute at the www site kazusa.or.jp/codon.

In some embodiments, an expression vector for use in the invention comprises an RbcL cDNA, e.g., a cDNA from a green microalgae such as a Chlamydomonas that encodes a functional RbcL protein, and an expression cassette comprising an RbcL gene promoter, e.g., RbcL gene accession number J01399); the first 90 nucleotides of the RbcL coding sequence, a microalgal chloroplast codon-optimized ADH gene and an RbcL terminator. The first 90 nucleotides of the RbcL coding sequence serves as a chloroplast expression enhancement sequence. In some embodiments, the chloroplast expression enhancement sequence comprises at least 30, 60, 75, 120, 150, or 180, or more nucleotides of the RbcL coding sequence. Upon introduction, double homologous recombination restores expression of functional Rubisco such that the microalgae can perform autotrophic photosynthesis. RbcL genes are known in the art. In some embodiments, the expression vector encodes an RbcL protein having at least 95% identity, or at least 96%, at least 97%, at least 98%, or at least 99% identity to the RbcL protein sequence of accession number AAA84449.1. In typical embodiments, very low levels, (e.g., (0%, 1%, 2%, or fewer), false positives during screening.

In some embodiments, the transgene encodes a yeast enzyme, e.g., a yeast alcohol dehydrogenase. In some embodiments, the transgene is a yeast ADH1 gene, e.g., accession no. YOL086C) that has been codon optimized for expression in microalgae. In some embodiments, pyruvate decarboxylase is expressed, e.g., along with an alcohol dehydrogenase. Thus, insome embodiments, the systems of the invention is used to produce ethanol. In some embodiments, expression of a transgene, e.g., an alcohol dehydrogenase and/or pyruvate decarboxylase results is increased about 30-100-fold relative to traditional methods, e.g., employing antibiotic selection.

In some embodiments, the transgene is an isoprene synthase gene, e.g., poplar or kudzu isoprene synthase that has been codon optimized for expression in microalgae. In some embodiments, expression of isoprene synthase is increased about 30-100-fold relative to traditional methods, e.g., employing antibiotic selection.

Cell transformation methods for cyanobacteria are well known in the art (Wirth, Mol Gen Genet. 1989 March; 216(1):175-7; Koksharova, Appl Microbiol Biotechnol 2002 February; 58(2): 123-37; Thelwell). Transformation methods and selectable markers for use in bacteria are well known (see, e.g., Sambrook et al., supra).

In microalgae, e.g., green microalgae, the nuclear, mitochondrial, and chloroplast genomes are transformed through a variety of known methods, including by microparticle bombardment, or using a glass bead method (see, e.g., Kindle, J Cell Biol 109:2589-601, 1989; Kindle, Proc Natl Acad Sci USA 87:1228-32, 1990; Kindle, Proc Natl Acad Sci USA 88:1721-5, 1991; Shimogawara, Genetics 148:1821-8, 1998; Boynton, Science 240:1534-8, 1988; Boynton, Methods Enzymol 264:279-96, 1996; Randolph-Anderson, Mol Gen Genet 236:235-44, 1993).

### EXAMPLES OF METHODS OF THE INVENTION

### Chlamydomonas Strains and Culture Media

Chlamydomonas reinhardtii strain CC2653 (Spreitzer et al., 1985), obtained from the Chlamydomonas Center (<http://www.chlamy.org>), was employed as the recipient strain for chloroplast transformation purposes. Strain CC2653 is a chloroplast mutant that contains a point mutation in the 5′ end of the RbcL gene coding region, causing an early termination of the RbcL protein synthesis (Spreitzer et al., 1985). In consequence, lack of the RbcL large subunit of the enzyme ribulose-1,5-bisphosphate carboxylase/oxygenase (Rubisco, EC4.1.1.39) led to the absence of both the chloroplast-encoded RbcL large subunit, and the nuclear-encoded RbcS small subunit of the Rubisco. Therefore, strain CC2653 is deficient in Rubisco and unable to grow photo-autotrophically. It requires acetate for growth, and is light sensitive. This strain was cultivated in a TRIS-Acetate-Phosphate (TAP) medium, pH 7.0 (Gorman and Levine, 1965), under dim light or in the dark. For other Chlamydomonas strains, cells were grown either photo-mixotrophically in TAP medium, or photo-autotrophically in HS minimal medium (Sueoka, 1960). Cells in liquid culture were grown in Erlenmeyer flasks at 24° C. with shaking under continuous illumination at approximately 50 μmol photons m⁻² s⁻¹. Culture density was measured by cell counting using a Neubauer ultraplane hemacytometer and a BH-2 light microscope (Olympus, Tokyo).

### Construction of Expression Vector for Chlamydomonas Chloroplast Transformation

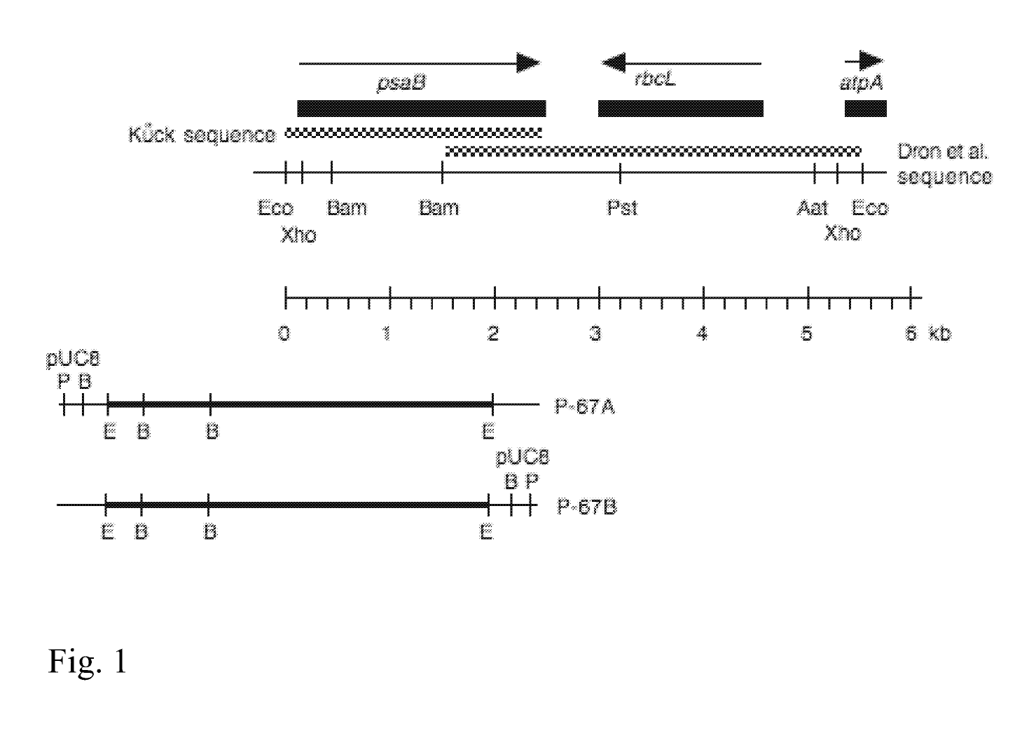

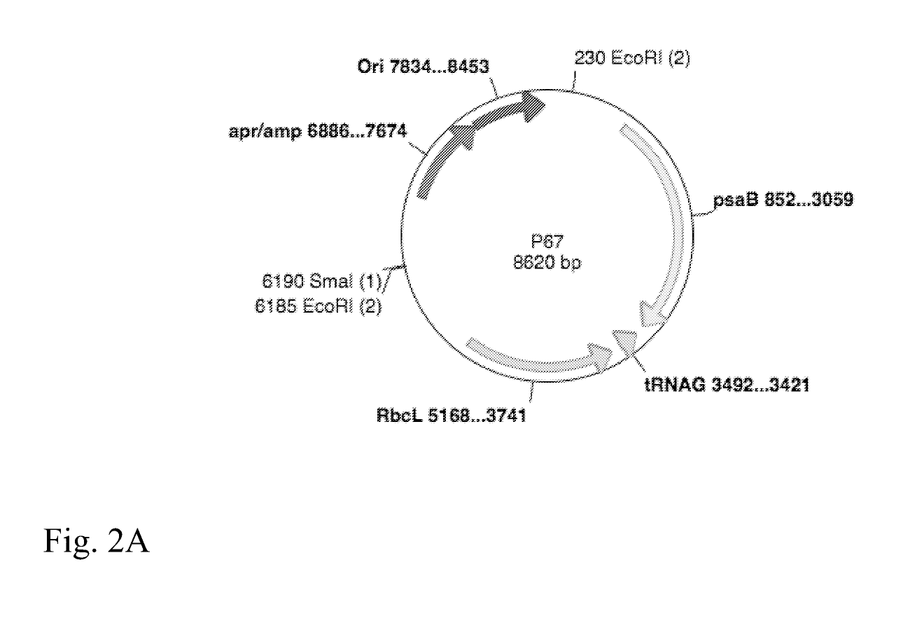

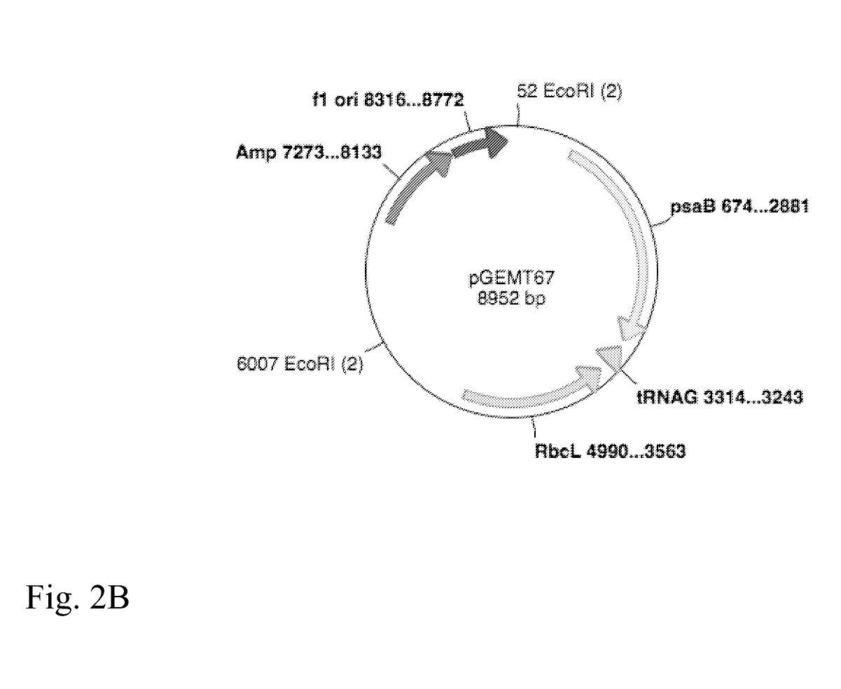

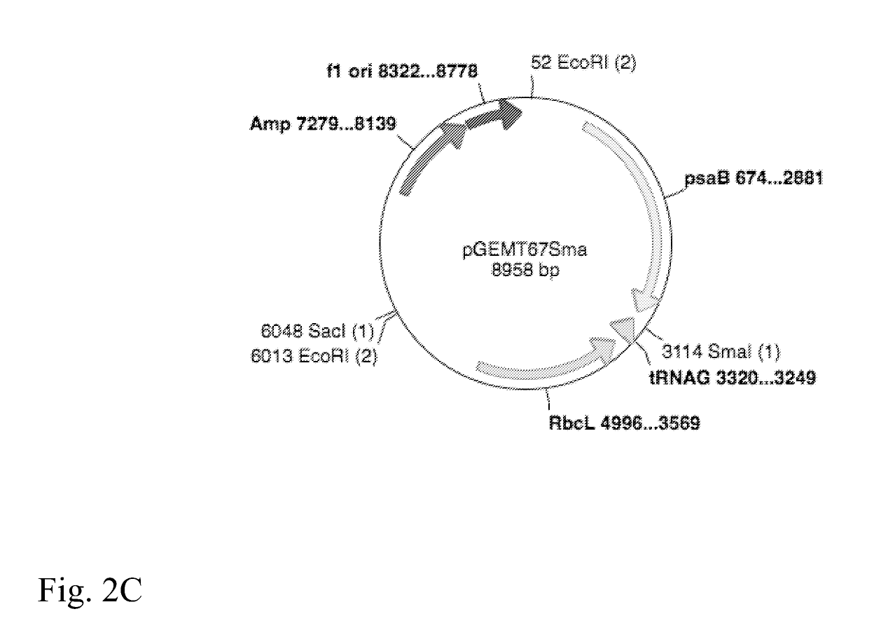

Plasmid P-67 containing the C. reinhardtii chloroplast DNA EcoRI 14 fragment (5.8 kb) was acquired from the Chlamydomonas Center, and used as the starting material for the construction of the expression vector. This EcoRI 5.8 kb DNA contains the PsaB, tRNAG, and RbcL genes, as well as the 5′ end of the atpA gene (FIG. 1). In this example, we used a functional copy of the RbcL gene from the EcoRI 14 fragment, in conjunction with the yeast ADH1 transgene, to simultaneously replace the mutated RbcL gene in the recipient strain CC2653 and to confer ADH1 expression, through double-homologous recombination upon suitable chloroplast transformation. Such replacement of the defective RbcL gene permits the selection of chloroplast transformants based on photoautotrophic growth phenotype. To build the expression vector, the EcoRI 5.8 kb insert was firstly isolated from P-67 plasmid (FIG. 2A) and cloned into the pGEMT-easy vector (Promega), generating plasmid pGEMT67 (FIG. 2B). Then, a unique SmaI restriction site was introduced in the intergenic region of PsaB and tRNAG genes through site-directed mutagenesis, generating plasmid pGEMT67Sma (FIG. 2C). Plasmid pGEMT67Sma was used as the backbone vector for the cloning of the ADH expression cassette.

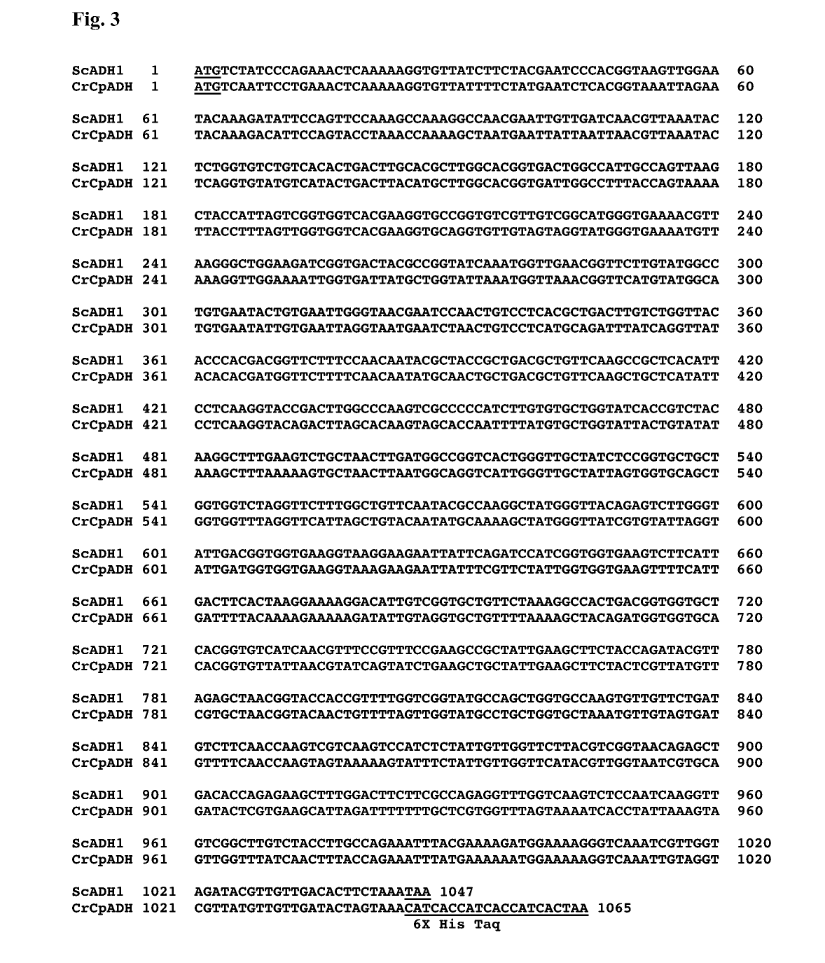

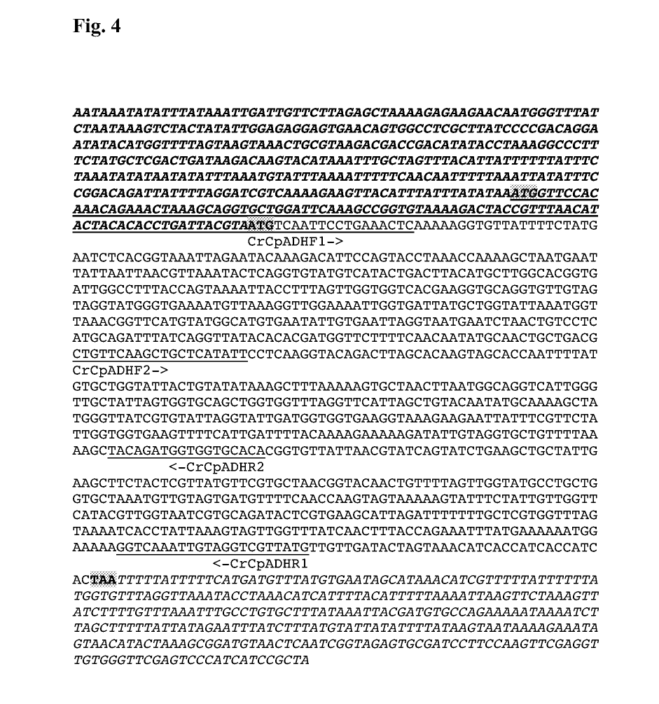

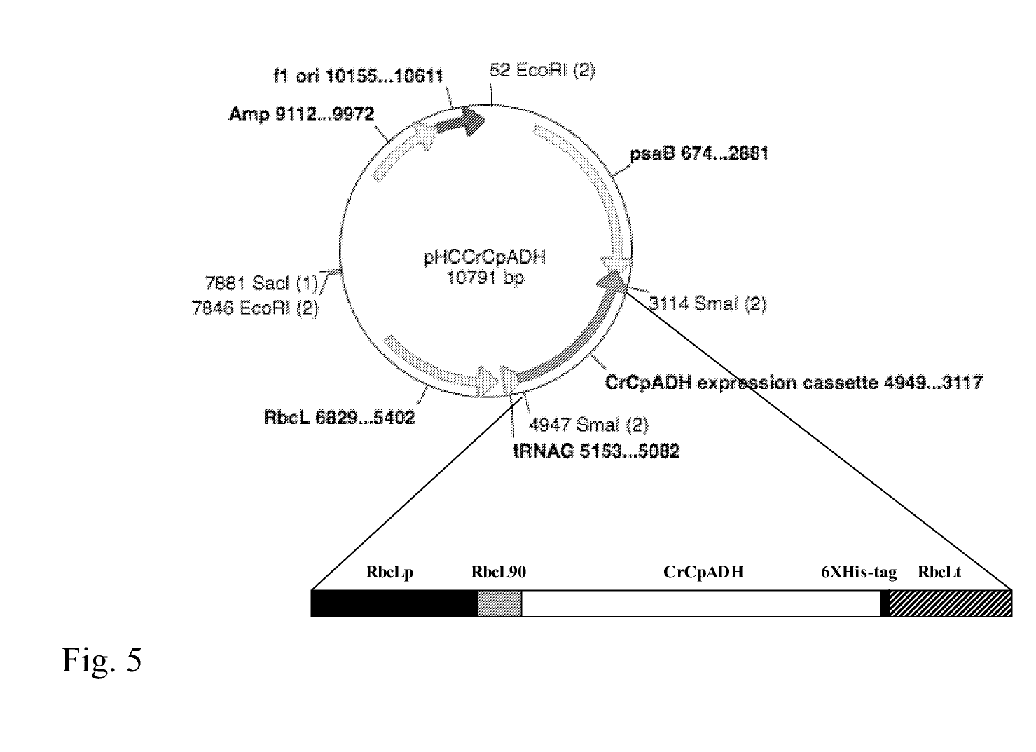

The ADH gene sequence for the transformation of the Chlamydomonas chloroplast was designed on the basis of the ADH1 gene of Saccharomyces cerevisiae (accession no. YOL086C). The sequence of the ADH1 gene was “codon-optimized” to match the Chlamydomonas chloroplast codon usage, so as to maximize the expression of the yeast ADH protein in the Chlamydomonas chloroplast. FIG. 3 shows the nucleotide alignment of the sequence from Saccharomyces cerevisiae ADH1 gene (ScADH1) and the codon-optimized sequence for Chlamydomonas reinhardtii for chloroplast transformation (CrCpADH). The translation initiation codon ATG, and the termination codon TAA are labeled in red and underlined font. The sequence encoding the 6×His-tag at the 3′ end of CrCpADH is also underlined. To confer expression of the ADH gene in the chloroplast of Chlamydomonas reinhardtii, we used the promoter of the RbcL gene (accession number J01399), encoding the large subunit of the ribulose-1,5-bisphosphate carboxylase/oxygenase. The promoter sequence used for the construction of the expression cassette includes the region of the RbcL gene from nucleotide −350 to +90 (the nucleotide “A” of the initiation codon ATG of the gene is considered as +1). An expression cassette comprising the RbcL gene promoter, the ADH codon-optimized DNA sequence, including a 6×His-tag at the 3′ end of the coding sequence, and the RbcL gene terminator-3′UTR (FIG. 4) was synthesized under contract by DNA 2.0 (Menlo Park, Calif.). The entire CrCpADH expression cassette was then inserted into plasmid pGEMT67Sma, in the intergenic region between PsaB and tRNAG genes. The resulting plasmid was termed pHCCrCpADH, the structure of which is shown in FIG. 5. The expression vector pHCCrCpADH was then used to transform the chloroplast of the Rubisco-less mutant CC2653 strain through a biolistic approach.

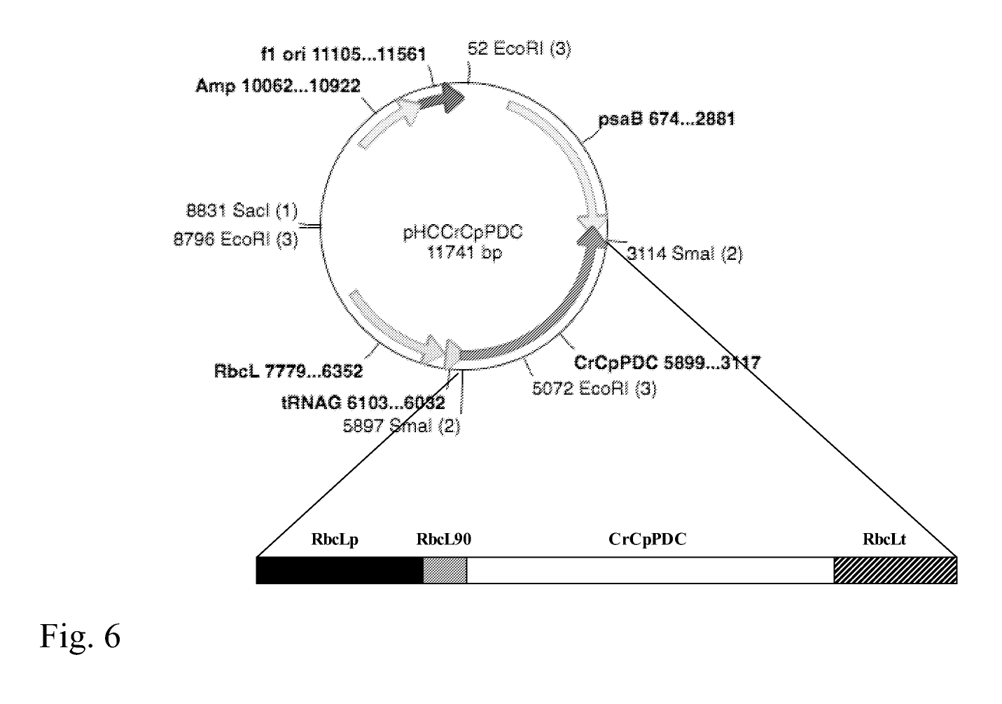

In a similar approach, the nucleotide sequence of the pyruvate decarboxylase PDC gene was codon-optimized to match the Chlamydomonas chloroplast codon usage, so as to maximize the expression of the pyruvate decarboxylase protein in the Chlamydomonas chloroplast (FIG. 6).

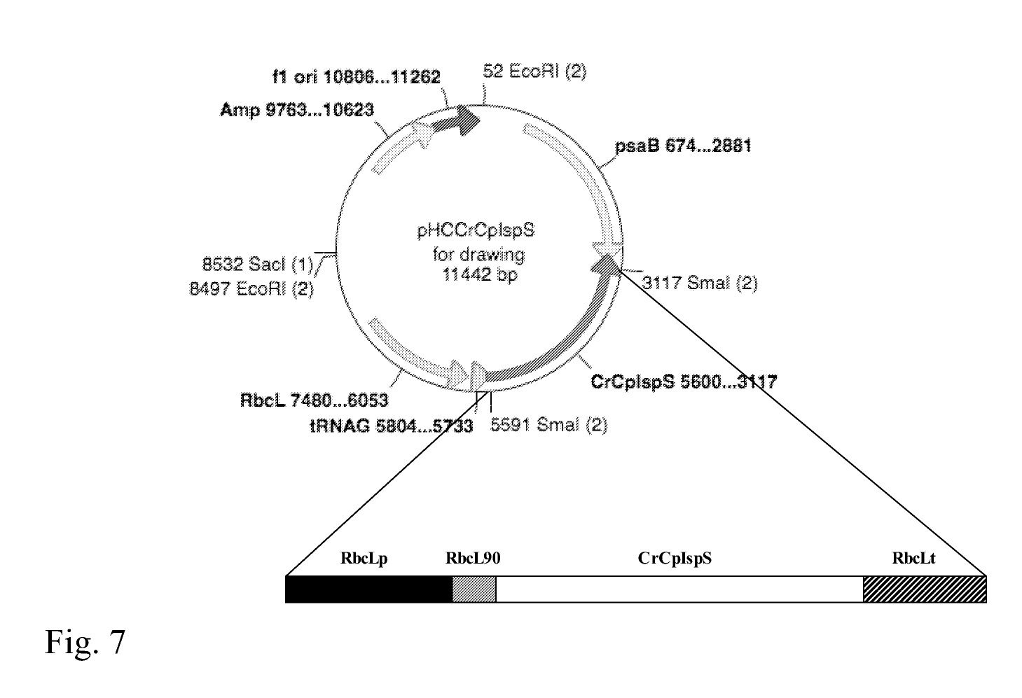

In yet another similar approach, the nucleotide sequence of the isoprene synthase IspS gene was codon-optimized to match the Chlamydomonas chloroplast codon usage, so as to maximize the expression of the isoprene synthase protein in the Chlamydomonas chloroplast (FIG. 7).

### Chloroplast Transformation and Selection

Chlamydomonas reinhardtii strain CC2653 was plated on 1.5% agar as a thin liquid layer of cells at a density of approximately 1−2×10⁷ cells per Petri dish (85 mm diameter) containing HS minimal medium. Gold particles (1 μm diameter) coated with pHCCrCpADH plasmid DNA were delivered into cells upon bombardment with a Biolistic PDS-1000/He Particle Delivery System (BioRad) operated at 1100 psi. Approximately 5 μg of plasmid DNA, linearized with SacI restriction enzyme (FIGS. 5, 6 and 7) was used in each bombardment. Double-homologous recombination (also referred to as crossover) occurred in the chloroplast between the PsaB and RbcL DNA sequences of strain CC2653 and the corresponding DNA sequences in plasmid pHCCrCpADH in essence replacing the PsaB-tRNAG-RbcL endogenous DNA fragment with the PsaB-CrCpADH-tRNAG-RbcL DNA piece from the plasmid. The double-homologous recombination, when successfully implemented, is expected to confer both autotrophy (functional RbcL gene) and ethanol production (ADH transgene) in the transformant CC2653 strains.

Following the biolistic treatment, plates were incubated at 24° C. under continuous illumination at about 50 μmol photons m⁻² s⁻¹. After two weeks incubation, transformant colonies became visible to the eye, indicating ability of autotrophic growth by the cells. Such individual colonies were transferred onto fresh HS minimal medium agar plates. Once such autotrophic strains established themselves (about two weeks following transfer of the transformants), cells from individual colonies were cultured in liquid HS minimal media for 2 days and re-plated onto HS agar plates to obtain individual single-cell lines. The resulting lines were then tested for the presence of the ADH gene and for homoplasmy of the chloroplast DNA by PCR analysis.

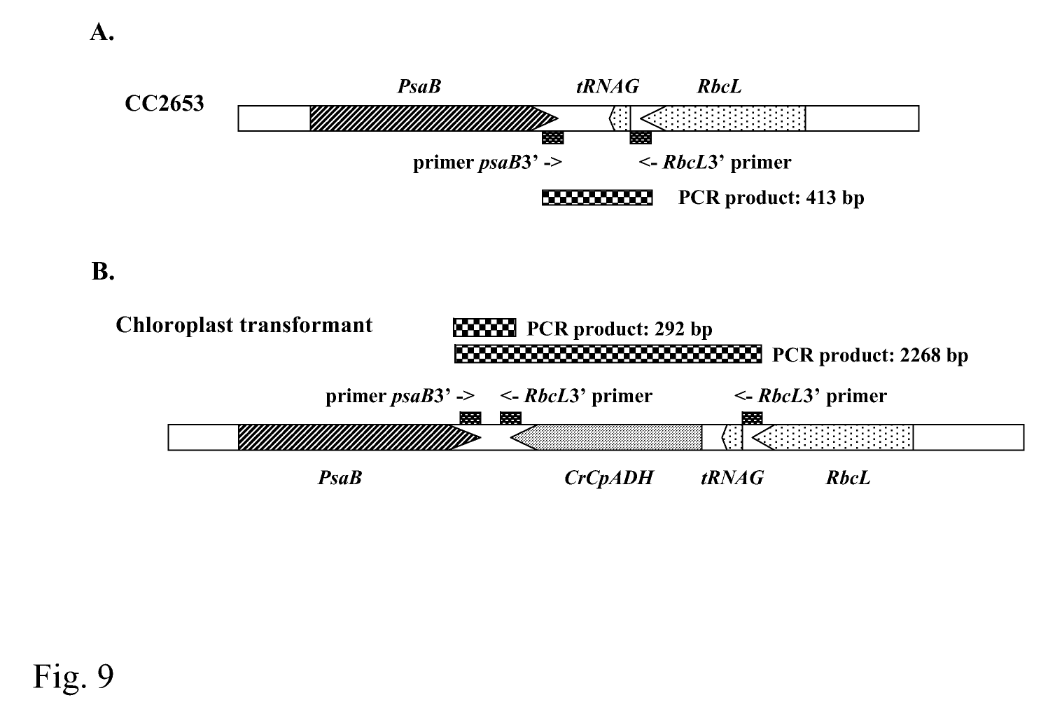

Presence of the transgenes in the transformants was tested upon PCR analysis by using the following set of ADH-specific primers: CrCpADHF2 5′CTGTTCAAGCTGCTCATATT3′, and CrCpADHR2 5′TACAGATGGTGGTGCACA3′, anticipating a fragment size of 324 bp (FIG. 4). Homoplasmy of the chloroplast DNA in the transformants was also tested by PCR analysis by using a pair of primers, one located in the 3′ UTR region of the PsaB gene with the sequence 5′-CTACCTCATCAGATCAGC-3′ (FIG. 9, primer psaB3), the other located at the 3′ end of RbcL gene with the sequence 5′-GGATGTAACTCAATCGGTAGAG-3′ (FIG. 9, RbcL3′ primer). This set of primers anticipated amplification of an endogenous fragment of 413 bp from the wild type DNA (FIG. 9A), whereas the size of the amplified fragment was anticipated to be 2268 bp in the pHCCrCpADH transformant strains. The larger size in the latter case would be due to the presence of the CrCpADH expression cassette in the intergenic region of the PsaB-tRNAG in the transformants. In addition, use of these primers projected amplification of a 292 bp fragment in the chloroplast transformants because the of “RbcL3′ primer” sequence is also present in the 3′ end of the expression cassette that employs the RbcL 3′UTR as a transcription terminator (FIGS. 5 and 9B). In these experiments, the PCR reaction time was limited to 30 seconds, which only allowed amplification of small DNA fragments such as the 413 bp or the 292 bp fragments, but not the 2268 bp nucleotide.

### Expression Analysis by RT-PCR

Total RNA was extracted with Trizol (Invitrogen) following the manufacturer's instructions. Amplification of ADH transcripts was performed as follows: 0.1 μg of total RNA was used for the reverse transcription with an ADH gene specific primer located at the 3′ end of the gene, termed CrCpADHR1: 5′CATAACGACCTACAATTTGACC3′, using Superscript III from Invitrogen and by following the manufacturer's instructions. The reaction mix was then diluted 3× and 1 μl aliquot was employed for the subsequent PCR reaction using the primers: CrCpADHF1 5′ATGTCAATTCCTGAAACTC3′ and CrCpADHR2 5′TGTGCACCACCATCTGTAGC3′ (FIG. 4) anticipating a product size of 720 bp. For the RT-PCR amplification of the RbcL gene transcripts, encoding the Rubisco large subunit (Accession No. J01399), 0.1 μg of total RNA was used for reverse transcription with an RbcL specific primer located at the 3′ end of the gene with the following sequence: 5′CTTCACATGCAGCAGCAAGTTCTGG3′. The reaction mix was then diluted 3× and 1 μl was used for the subsequent PCR reaction using primers: RbcL forward 5′ GCCGGTGTAAAAGACTACCG 3′ and RbcL reverse 5′ CGTGTGGAGGACCTACGAAT 3′, anticipating a PCR product of 418 bp.

### Protein Analysis by Western Blotting

Cells were harvested by centrifugation at 3000 g for 5 min at 4° C., and cell pellets were resuspended in ice-cold sonication buffer containing 50 mM Tricine (pH 7.8), 10 mM NaCl, 5 mM MgCl₂, 0.2% polyvinylpyrrolidone-40, 0.2% sodium ascorbate, 1 mM aminocaproic acid, 1 mM aminobenzamidine and 100 μM phenylmethylsulfonylfluoride (PMSF). Cells were broken by sonication in a Branson 250 Cell Disruptor operated at 4° C. for 30 s (pulse mode, 50% duty cycle, output power 5). For total protein extraction, an equal volume of 2× protein solubilization buffer containing 0.5 M Tris-HCl (pH 6.8), 7% SDS, 20% glycerol, 2 M urea, and 10% β-mercaptoethanol was added.

Soluble and membrane protein fractions were separated as follows: the sonicated suspension was centrifuged at 10,000 g for 5 min at 4° C. to pellet the membrane fraction. The supernatant containing mostly soluble proteins was concentrated by Amicon centrifugal filter devices 10K (Millipore) and resuspended in solubilization buffer at a concentration of 1 μg protein per μl. Membrane fractions were resuspended in solubilization buffer on the basis of equal chlorophyll concentration. The latter was determined from the absorbance of a pigment extract in 80% acetone spectrophotometrically (Amon 1949). Solubilized protein extracts were resolved by SDS-PAGE using the discontinuous buffer system of Laemmli (1970). After completion of the electrophoresis, proteins on polyacrylamide gels were either stained with Coomassie Brilliant Blue or electro-transferred onto PVDF membrane Immunoblot analysis was carried out with specific polyclonal or monoclonal antibodies, followed by chemiluminescence detection of the cross reactions using the SuperSignal West Pico Chemiluminesencet substrate (Pierce-Thermo Scientific).

### Ethanol Production Measurements

Cells were grown to the mid exponential growth phase (OD₇₃₀=0.2-0.3, i.e., about 2−3×10⁶ cells/ml), in Erlenmeyer flasks at 24° C. upon orbital shaking under continuous illumination of about 50 μmol photons m⁻² s⁻¹. The culture was then centrifuged and cell pellet was resuspended in fresh TAP medium at a concentration of OD₇₃₀=0.9. The concentrated culture was then sealed and incubated at 24° C. under either light-dark cycles (LD=12 h light, 12 h dark) or in total darkness (D) for 48 h. The ethanol content of the liquid medium in the culture was quantified using a dichromate-based method (Ethanol Assay kit from the BioChain Institute, Inc., Hayward, Calif., USA). To prepare the liquid samples for this assay, cells were first pelleted from the growth medium, and the supernatant was filtered through a nylon syringe filter of 0.45 μm pore size (National Scientific) to remove insoluble particles prior to measuring the ethanol content of the liquid phase.

### Isoprene Production Measurements

Volatile isoprene hydrocarbons accumulation was measured from the gaseous composition of the culture headspace. Gas from the headspace of sealed cultures was sampled and analyzed by gas chromatography using a Shimadzu 8A GC (Shimadzu, Columbia, Md., USA) equipped with a flame ionization detector (FID) and a column selected to detect short-chain hydrocarbons. Amounts of isoprene produced were estimated by comparison with a pure isoprene standard (Acros Organics, Fair Lawn, N.J., USA).

### Construction of Expression Vector for Chlamydomonas Reinhardtii Chloroplast Transformation

The complete nucleotide sequence of the CrCpADH expression cassette is shown in FIG. 4. This construct includes the RbcL promoter (FIG. 4, blue font) including the first 90 nucleotides of the RbcL coding region, with the ATG start codon of the RbcL underlined. This is followed by the CrCp codon-optimized ADH DNA sequence (in black font). Start ATG and stop TAA codons for the CrCpADH gene are shown in red underlined font. The construct also includes a 6×His tag at the 3′ end of the ADH coding sequence, immediately preceding the TAA stop codon (underlined). Lastly, the RbcL terminator (FIG. 4, red font nucleotides) was placed at the 3′ end of the construct. Also shown in FIG. 4 is the position of CrCpADH forward (F1, F2) and reverse primers (R1, R2) employed in this work.

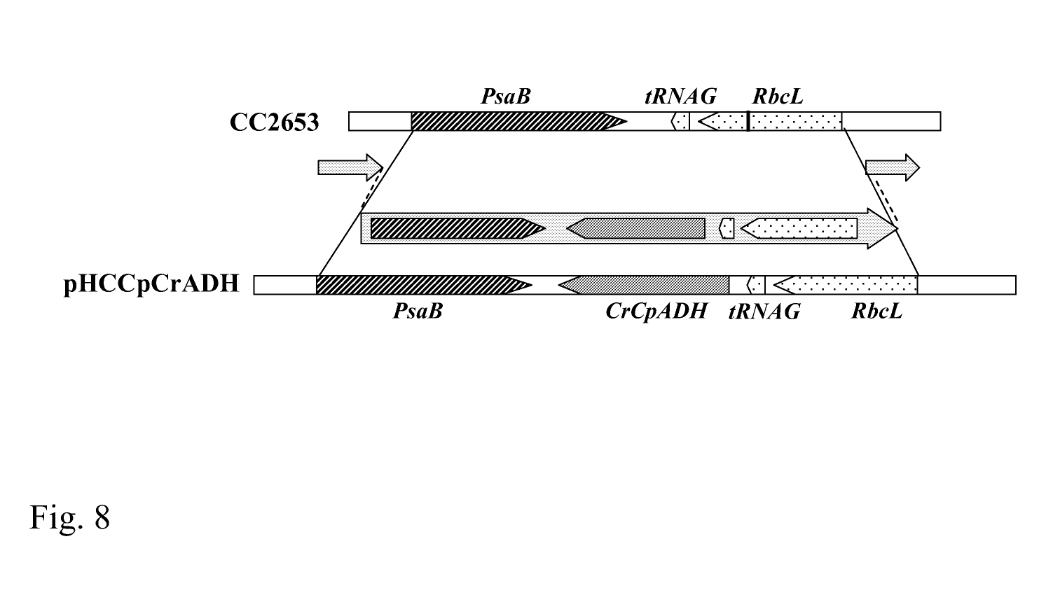

The expression vector pHCCrCpADH having the CrCpADH cassette inserted in the intergenic region of PsaB-tRNAG (FIG. 5) was used to transform the chloroplast of the Rubisco-less mutant CC2653 strain through a biolistic approach. Following biolistic delivery of the DNA construct to the Chlamydomonas chloroplast, the CrCpADH expression cassette was integrated into the chloroplast genome via double homologous recombination mediated through a crossover at the PsaB and tRNAG-RbcL DNA regions (FIG. 8). The occurrence of homologous recombination permitted replacement of the native mutant and non-functional RbcL gene of the recipient strain by a functional copy of the RbcL gene located in the pHCCrCpADH expression vector. Integration of a functional copy of the RbcL gene in the recipient CC2653 strain conferred photoautotrophic growth to the transformants. This “recovery of function” property was used as the selectable marker for the isolation of successful chloroplast transformants.

The “recovery of function” selection criterion employed in this invention is superior to that of an antibiotic resistance based selection, because (i) it substantially minimizes the recovery of false positives, (ii) accelerates the process of achieving chloroplast DNA homoplasmy without the otherwise required persistent use of antibiotics, and (iii) alleviates concerns of undesirable secondary mutagenesis effects, which are common side effects many antibiotics have on microorganisms (Harris, 1989). For example, in our “recovery of function” based selection, more than 50 putative transformants were isolated from each transformation plate, based on the photoautotrophic growth criterion. All of these isolated putative transformant strains were true positive, i.e., they were shown to contain the CrCpADH transgene (see below).

### Analysis of Chloroplast Transformants

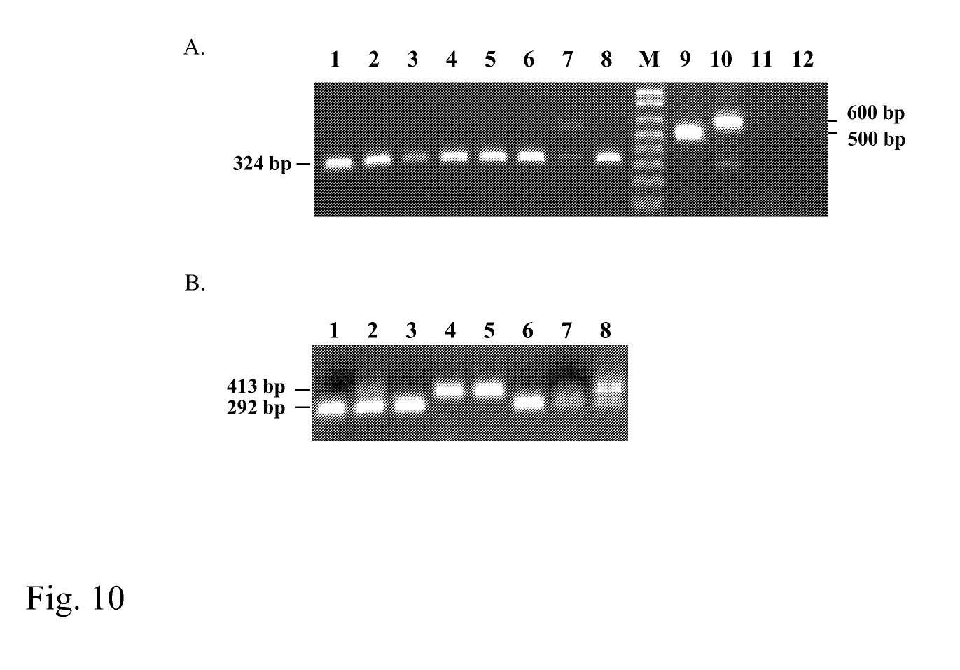

Eight independent and randomly selected CrCpADH transformant lines that grew under selective photoautotrophic conditions (function of the RbcL gene) were isolated for further testing. These putative transformants were first tested for the presence of the ADH gene via genomic PCR analysis. Probing with ADH gene specific primers (see Materials and methods) resulted in positive amplification of the anticipated 324 bp product size of ADH gene sequence in all randomly selected transformants (FIG. 10A, lanes 1-8), although PCR results for lanes 3 and 8 were somewhat uncertain, possibly due to the lower quality of the DNA sample used. As control PCR analysis, genomic DNA was also employed from two non-transformed strains, the recipient CC2653 strain, used for the chloroplast transformation in this work, and another wild type strain (CC2918), by using the same set of primers for PCR analysis. The control strains showed no amplification of ADH specific sequence (FIG. 10A, lane 11 for CC2653 and FIG. 10A, lane 12 for CC2918). To further increase confidence that lack of ADH gene sequence amplification in the control CC2653 and CC2918 strains is not due to DNA preparation from these samples, “mating type” specific primers were used in a separate PCR experiment to test the quality of these DNA samples. Both CC2653 and CC2918 control strains showed the anticipated PCR products, i.e., 500 bp for the mating type “plus” CC2653 (FIG. 10A, lane 9) and 600 bp for the mating type “minus” CC2918 (FIG. 10A, lane 10).

Chloroplast DNA homoplasmy in the CrCpADH transformants was also assessed by chloroplast DNA PCR. The issue here is to assess whether the transgenic DNA copy that carries the CrCpADH transgene has quantitatively replaced all (about 100) wild type copies of the chloroplast DNA in the isolated transformant cell lines. Achieving homoplasmy on the basis of an antibiotic selectable marker is often difficult, as antibiotics are not essential for cell growth and chloroplast DNA multiplication, whereas “recovery of function” would be key to the basic cell and chloroplast functions. Accordingly, “recovery of function” may be a more powerful condition favoring transgene chloroplast DNA segregation. The position of primers used to test homoplasmy of the chloroplast DNA in the CrCpADH transformants is shown in FIG. 4 (see also Materials and methods). Results from this analysis are shown in FIG. 10B. Lanes 1, 3 and 6 showed amplified PCR products of 292 bp that could only be derived from the “transgenic” chloroplast DNA (FIG. 9). Conversely, FIG. 10B, lanes 4 and 5 showed an amplified PCR product of 413 bp that corresponds to amplification of the “native” chloroplast sequence (FIG. 9). This analysis also showed that lanes 2 and 8 (FIG. 10B) contained a mixture of products derived from both the “native” and “transgenic” chloroplast DNA. It is concluded that replacement of the (up to 100) wild type copies of the chloroplast DNA by the transgenic copy in the transformant Chlamydomonas is not spontaneous, even under conditions when the transforming plasmid confers recovery of an essential function to the chloroplast.

Based on the above homoplasmy testing, a rigorous screening of numerous CrCpADH transformant strains was undertaken. As a result, we selected eight independent homoplasmic lines of chloroplast transformants for further gene expression analysis.

Analysis of ADH Gene Expression in Chlamydomonas reinhardtii Chloroplast

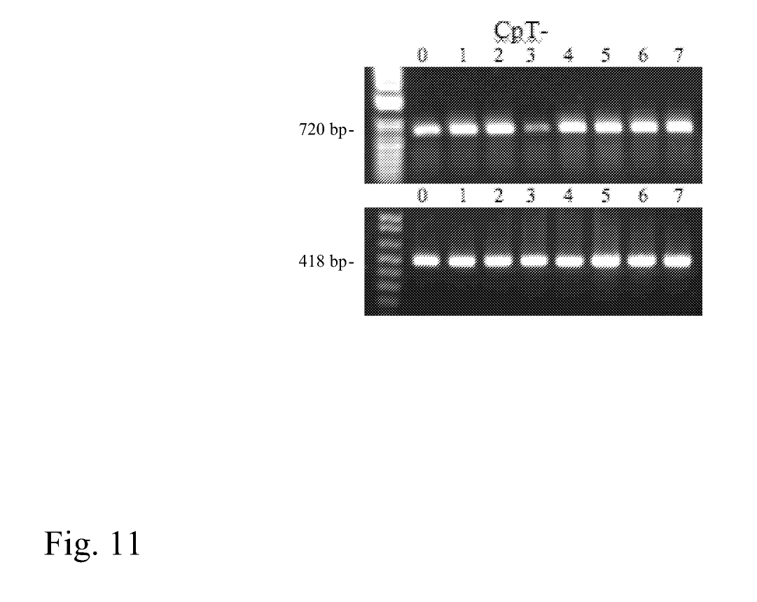

The steady state levels of ADH transcripts were examined by RT-PCR in eight isolated and verified homoplasmic lines of chloroplast transformants (lines CpT-0 through CpT-7). Using CrCpADH gene specific primers, strong expression of the ADH gene was evidenced by the presence of a 720 bp product in all but the CpT-3 line (FIG. 11, upper panel). FIG. 11 (lower panel) shows the RT-PCR result with RbcL gene specific primers, as a control of endogenous chloroplast gene expression. In this case, similar levels of amplification were observed in all isolated transformant lines indicating that similar starting cDNA templates were used among these lines. It is noted that the expression level of CrCpADH is similar to that of RbcL, which is known to be one of the highest expressed genes in Chlamydomonas.

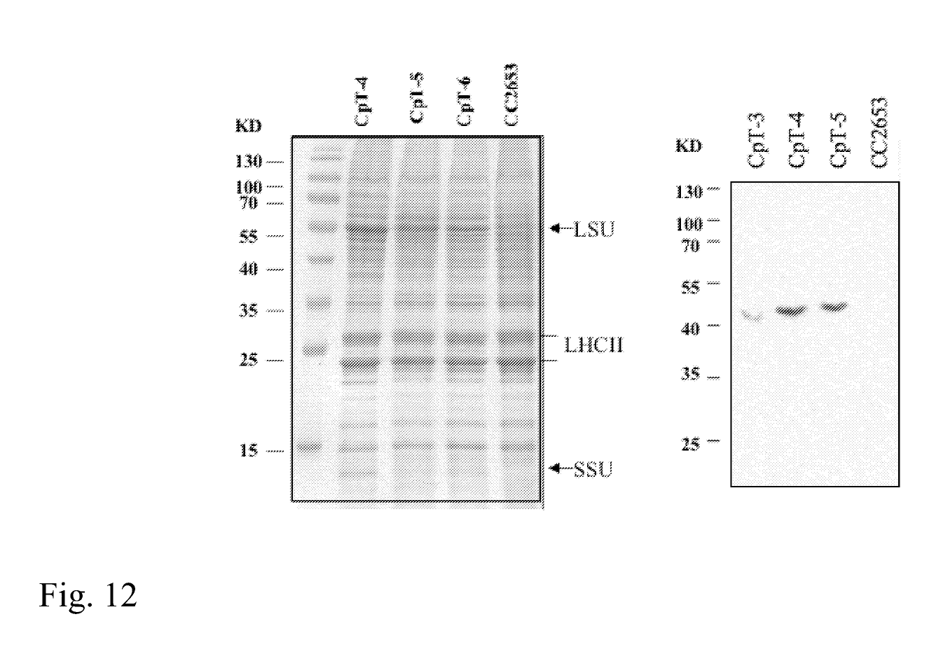

Presence of the ADH protein was also tested by Western blot analysis. FIG. 12 shows the Coomassie-blue stained SDS-PAGE of total protein extract of three independent transformant lines (CpT-4, CpT-5 and CpT-6) and that of the recipient CC2653 strain. It is clearly seen that CpT transformants accumulate both the large (LSU) and small (SSU) subunits of Rubisco, whereas the recipient strain CC2653 lacks both of these subunits of the Rubisco protein. This is consistent with the autotrophic property of the transformants, as they have acquired a functional RbcL gene from the transforming plasmid and apparently assemble a function Rubisco (FIG. 12). It is also of interest to note that other chloroplast proteins were not affected either by the CC2653 mutation (White and Melis 1997) or by the transformation of the recipient strain by the CrCpADH transgene.

Presence of the ADH protein in the transformants was also tested by Western blot analysis, using specific monoclonal antibodies against the 6×His-tag (Invitrogen, USA). Evidence was obtained to show a specific cross-reaction between the 6×His monoclonal antibodies with a protein of approximately 43 kD (FIG. 12, right panel, Cp-T lanes). No such cross-reaction could be detected in the recipient strain (CC2653) suggesting specificity of ADH expression in the transformants but not in the control.

### Ethanol Production Analysis

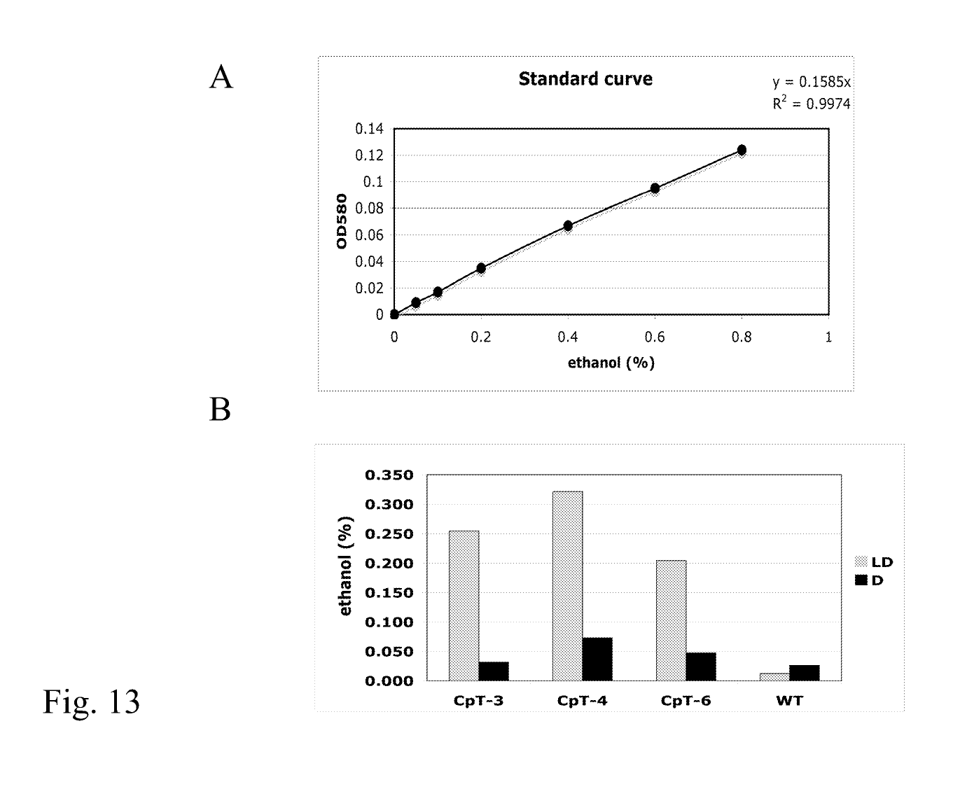

Ethanol concentration calibration curves were obtained with the dichromate-based method (Ethanol Assay kit from BioChain, see Materials and methods) by following the manufacturer's specifications. A representative ethanol calibration standard curve (FIG. 13) showed a linearity of R²≧0.99, with an equation of Y=0.16×, where X is the ethanol concentration in percent (w/v) and Y is the absorbance of the ethanol assay reaction products at 580 nm.

For ethanol production measurements, C. reinhardtii CrCpADH transformant and wild type strains were grown to a similar cell density (OD₇₃₀=0.2-0.3) and subsequently concentrated to a higher cell density (OD₇₃₀=0.9) before being subjected to various treatments and incubations for ethanol production. At the end of the respective incubation, cells were pelleted from their suspension and supernatants were filtered through a 0.45 μm syringe filter prior to assaying the ethanol content of the liquid phase. his centrifugation and filtering pretreatment was necessary in order to eliminate scattering effects by insoluble matter in the samples, as scattering interfered with the spectrophotometric quantitation measurements. Even with these precautions, some variations in ethanol productivity among different samples in separate experiments were noted, as shown in FIG. 13B. Regardless of the variations in measured ethanol content, it was evident that C. reinhardtii transformant strains performed consistently better than the wild type control, especially under LD conditions. The highest ethanol content was measured with transformant line #4 (CpT-4), which reached an ethanol concentration level of 0.3% w/v under LD conditions. This was a 30-fold greater level of ethanol concentration as that measured with the wild type control under otherwise identical experimental conditions. It should be noted that the recipient CC2653 strain, from which the chloroplast transformants were generated, could not be tested in this assay, as it could not grow well under the ethanol production conditions employed. Hence, the control was replaced in these measurements with a wild type autotrophic strain, used as a control for ethanol production measurements.

### Isoprene Production Analysis

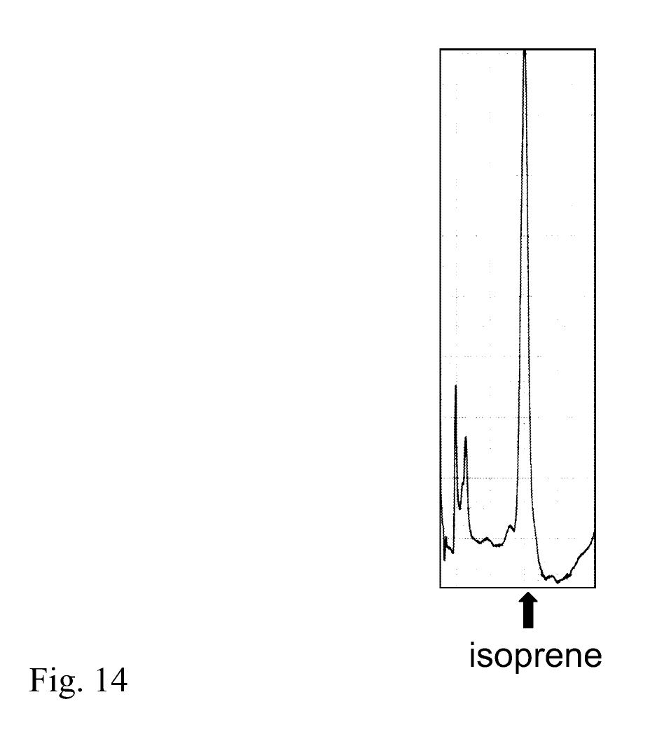

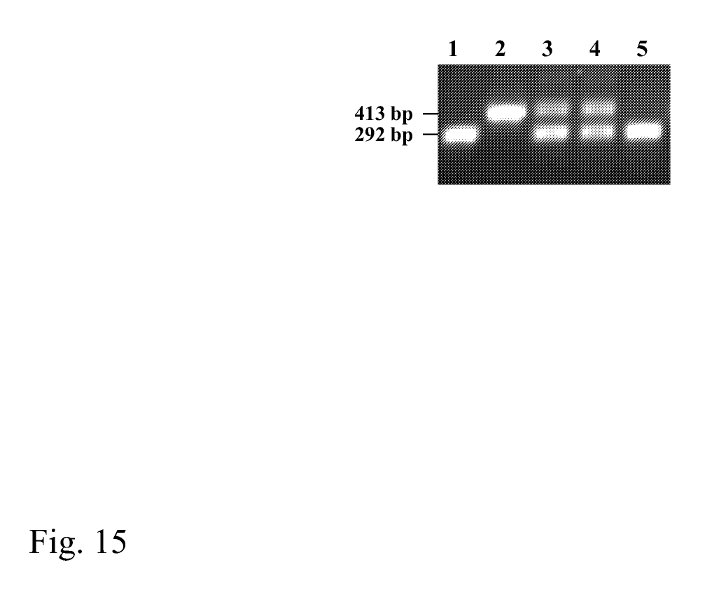

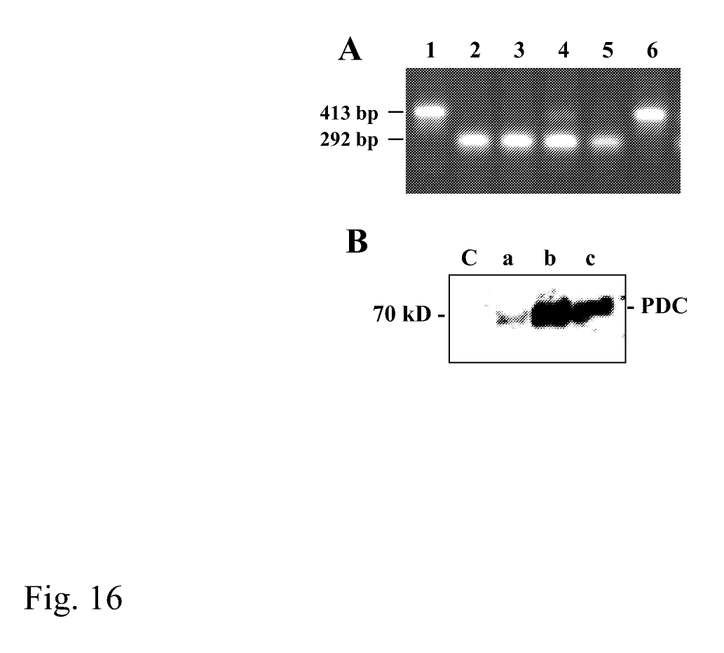

In all CrCpIspS transformants (FIG. 7), but not in their wild type counterparts, an isoprene-specific peak was clearly evident (FIG. 14), showing that the expressed enzyme is indeed active in the IspS transformant strains. FIG. 14 shows an example of a GC compositional analysis of headspace gases from photosynthetically grown CrCpIspS-transformed Chlamydomonas reinhardtii, showing the isoprene peak at a retention time of about 5 min after sample injection in the GC column. The identity of isoprene in the corresponding GC peak was established by comparison with an isoprene standard (Lindberg et al. 2010).

### Discussion

Earlier studies from different laboratories showed that Chlamydomonas reinhardtii does not produce ethanol in the light (Gfeller and Gibbs, 1984). However, ethanol production was detected under certain experimental conditions, including sulfur deprivation and hydrogen production (Winkler et al. 2002) and obligate anaerobiosis (Mus et al. 2007). Results in this disclosure are consistent with these earlier studies showing that the ethanol level produced under LD conditions was about half or less than that produced in the dark by the wild type control strains (FIG. 13B). On the contrary, ethanol production profiles of this green microalga transformed with the CrCpADH construct (FIG. 5) were much higher under LD conditions than in the dark (FIG. 13B). These results strongly suggest activation of an ethanol production pathway under illumination conditions specifically in the CrCpADH transformants, which is apparently due to the specific expression of the transgenic ADH gene in the latter.

Similarly, Chlamydomonas reinhardtii and other microalgae are not endowed with gene that confer upon them volatile isoprene hydrocarbons production. However, substantial isoprene production was detected upon transformation of this green microalga with the CrCpIspS construct (FIG. 7).

Two approaches could be applied when aiming at transgene expression in the chloroplasts of Chlamydomonas. One is through the nuclear transformation process to integrate into the nuclear genome of Chlamydomonas a transgene that contains a chloroplast transit peptide. The transgenic protein synthesized in the cytosol could then be targeted into chloroplast through the function of the transit peptide. The other approach is the chloroplast transformation method that we employed in the illustrative examples here, which allows the direct integration of the transgene into the chloroplast genome, allowing direct synthesis of the transgenic protein in the chloroplast without going through the protein import process. The second method has advantages over the first one in many aspects:

(1) The mode of integration of a transgene into Chlamydomonas nuclear genome is at random, which usually leads to significant variations in the levels of transgene expression. Epigenetic gene silencing phenomena often render this insertional transformation ineffective, if not useless. In consequence, a screening process of a large population of transformants is necessary in order to obtain a transgenic line that shows optimal expression level.

(2) In contrast, integration of transgenes into the chloroplast DNA is mediated through homologous recombination. Therefore, one can target the insertion sites in the area of the genome that contain highly expressed genes or “hot spots”, to assure the expression of the transgenes.

(3) Direct chloroplast transformation ensures the final localization of the protein by the direct protein synthesis in designated organelle.

(4) Chlamydomonas chloroplast contains over one hundred copies of chloroplast genome. Direct chloroplast transformation allows the initially inserted single copy transgene to multiply to the same copy numbers of the chloroplast genome after the transformation through the establishment of homoplasmy process. The high copy number of the transgene in the chloroplast thus ensures higher levels of expression.

### REFERENCES

Gfeller R P and Gibbs M (1984) Fermentative Metabolism of Chlamydomonas reinhardtii. I. Analysis of Fermentative Products from Starch in Dark and Light. Plant Physiology 75: 212-218
Gorman D S and Levine R P (1965) Cytochrome f and plastocyanin: their sequence in the photosynthetic electron transport chain of Chlamydomonas reinhardtii. Proceedings of the National Academy of Sciences of the United States of America 54: 1665-1669
Kasai S, Yoshimura S, Ishikura K, Takaoka Y, Kobayashi K, Kato K, Shinmyo A (2003) Effect of coding regions on chloroplast gene expression in Chlamydomonas reinhardtii. J Biosci Bioeng 95: 276-282
Lindberg P, Park S, Melis A (2010) Engineering a platform for photosynthetic isoprene production in cyanobacteria, using Synechocystis as the model organism. Metabol Engin 12:70-79
Michelet L, Lefebvre-Legendre L, Burr S E, Rochaix J D, Goldschmidt-Clermont M. (2010) Enhanced chloroplast transgene expression in a nuclear mutant of Chlamydomonas. Plant Biotechnol J. 2010 Aug. 31. [Epub ahead of print] DOI: 10.1111/j.1467-7652.2010.00564.x
Mus F, Dubini A, Seibert M, Posewitz M C, Grossman A R (2007) Anaerobic acclimation in Chlamydomonas reinhardtii: anoxic gene expression, hydrogenase induction, and metabolic pathways. J Biol. Chem. 282(35):25475-86
Spreitzer R J, Goldschmidt-Clermont M, Rahire M, Rochaix J D (1985) Nonsense mutations in the Chlamydomonas chloroplast gene that codes for the large subunit of ribulose-bisphosphate carboxylase/oxygenase. Proc Natl Acad Sci USA 82: 5460-5464
Sueoka N (1960) Mitotic Replication of Deoxyribonucleic Acid in Chlamydomonas reinhardtii. Proc Natl Acad Sci USA 46: 83-91
Winkler M, Hemschemeier A, Gotor C, Melis A and Happe T (2002) [Fe]-hydrogenases in green algae: photo-fermentation and hydrogen evolution under sulfur deprivation. Intl. J. Hydrogen Energy 27: 1431-1439

1. A method of selecting microalgal cells transformed with a transgene of interest, the method comprising:
introducing an expression vector into a population of microalgal cells having a lethal mutation in an RbcL photosynthetic gene, wherein the expression vector has an expression cassette comprising a transgene of interest to be expressed in the microalgal cells operably linked to a promoter, and has a polynucleotide comprising a RbcL gene, or a fragment thereof, that encodes an RbcL gene that restores Rubisco function in photosynthesis;
wherein introduction of the expression vector into the microalgal cells provides functional Rubisco;
culturing the microalgal cells under photoautotrophic growth conditions in minimal media lacking organic carbon supplementation;
selecting colonies that grow under the culture conditions; and
evaluating transgene expression in the selected colonies and identifying colonies that express the trangene, thereby selecting microalgal cells transformed with the transgene of interest.

2. The method of claim 1, wherein the polynucleotide comprises a cDNA encoding the RbcL photosynthetic gene.

3. The method of claim 1, wherein the step of culturing the microalgal cells is performed in the absence of antibiotic selective pressure.

4. The method of claim 1, wherein the transgene encodes alcohol dehydrogenase.

5. The method of claim 4, wherein the microalgae cells express pyruvate decarboxylase.

6. The method of claim 1, wherein the transgene encodes pyruvate decarboxylase.

7. The method of claim 1, wherein the expression vector comprises a second transgene to be expressed.

8. The method of claim 7, wherein the expression vector comprises alcohol dehydrogenase and pyruvate decarboxylase transgenes capable of being expressed in the microalgae cells.

9. The method of claim 7, wherein the first and second transgenes are operably linked to the same promoter.

10. The method of claim 7, wherein the second transgene is operably linked to a second promoter.

11. The method of claim 1, wherein the transgene encodes isoprene synthase.

12. The method of claim 1 further comprising culturing a colony selected in the selecting step under photautotrophic growth conditions in minimal media until homoplasmy of the chloroplast DNA is achieved.

13. The method of claim 1 wherein the microalgal cells are green microalgae.

14. The method of claim 1 wherein the transgene expression cassette comprises the RbcL gene promoter, the first 90 nucleotides of the RbcL coding sequence, a microalgal chloroplast codon-optimized ADH gene and an RbcL terminator.

15. The method of claim 1 wherein the microalgal cells are cyanobacteria.

16. A microalgae cell comprising a heterologous transgene of interest wherein the microalgae cell does not comprise an antibiotic resistance gene.

17. The microalgae cell of claim 16, wherein the microalgae cell has a lethal mutation in an RbcL photosynthetic gene.

18. The microalgae cell of claim 16, wherein the microalgae cells are green microalgae.

19. The microalgae cell of claim 16, wherein the microalgae cells are cyanobacteria.

20. The microalgae cell of claim 16, wherein the heterologous transgene encodes isoprene synthase.

21. The microalgae cell of claim 16, wherein the heterologous transgene encodes alcohol dehydrogenase.
